SEQUENCE LISTING (1 sequence)

SEQ ID NO: 1 - DNA, 1536 bp, Artificial Sequence
  polynucleotide encoding VSV-G protein
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

(19) United States
(12) Patent Application Publication
(10) Pub. No.: US 2018/0028600 A1
(43) Pub. Date: Feb. 1, 2018

(54) NOVEL RECOMBINANT EXOSOME AND USE THEREOF

(71) Applicant: KOREA INSTITUTE OF SCIENCE AND TECHNOLOGY, Seoul (KR)

(72) Inventors: Yeon-Sun HONG, Seoul (KR); Yoo Soo YANG, Seoul (KR); Cherl Hyun JEONG, Seoul (KR); In-San KIM, Seoul (KR)

(73) Assignee: KOREA INSTITUTE OF SCIENCE AND TECHNOLOGY, Seoul (KR)

(21) Appl. No.: 15/650,272

(22) Filed: Jul. 14, 2017

(30) Foreign Application Priority Data
Jul. 15, 2016 (KR) 10-2016-0090232

(51) Int. Cl. A61K 38/16; A61K 45/06; A61K 9/127

(52) U.S. Cl. CPC A61K 38/162; A61K 9/1271; A61K 45/06

(57) ABSTRACT

Provided is a recombinant exosome, and more specifically, to a recombinant exosome obtained from a eukaryotic cell transfected for the expression of a glucose transporter protein (GLUT) and a membrane fusogenic protein, and use thereof.

### CROSS-REFERENCE TO RELATED APPLICATION

This application claims priority to Korean Patent Application No. 10-2016-0090232 filed on Jul. 15, 2016 and all the benefits accruing therefrom under 35 U.S.C. §119, the contents of which are incorporated by reference in their entirety.

### REFERENCE TO SEQUENCE LISTING SUBMITTED VIA EFS-WEB

This application includes an electronically submitted sequence listing in .txt format. The .txt file contains a sequence listing entitled “2017-09-28_SeqListing_6877-0101PUS1.txt” created on Sep. 25, 2017 and is 2,503 bytes in size. The sequence listing contained in this .txt file is part of the specification and is hereby incorporated by reference herein in its entirety.

### BACKGROUND

The present disclosure relates to a novel recombinant exosome and use thereof.

Over the past decade therapeutic proteins have represented a leading breakthrough in medicine, and more than 130 proteins have been approved for clinical applications. The intracellular delivery of functional proteins offers a potential tool to replace missing, dysfunctional or poorly expressed proteins and/or antagonize key intracellular pathways. Despite the continued evolution of therapeutic proteins, the application of protein-based therapeutics has been limited by the need for therapeutic vehicles capable of delivering membrane proteins to the plasma membrane. Membrane protein defects contribute to numerous human disorders by causing problems in the regulation, transport of materials, or cellular integration of tissues. Given the immense complexity of natural membranes, the study of membrane proteins requires their reconstitution in an artificial membrane. Several lipid-vesicle systems have been described for the delivery of membrane proteins, however, it has proven difficult to find appropriate conditions that support the insertion of biologically active membrane proteins into artificial vesicles (Liguori et al., Expert Rev. Proteomics, 4: 79-90, 2007; Liguori et al., J. Control. Release 126: 217-227, 2008; Biner et al., FEBS Lett. 590: 2051-2062, 2016). Accordingly, these drawbacks motivate the exploration of alternative strategies to control functions of biologically and/or medically important proteins.

In the modern society, the prevalence of metabolic diseases represented by obesity and diabetes is rapidly increasing. According to a report by the World Health Organization, about 400 million people worldwide are obese as of 2006 and the National Diabetes Statistics Report of 2011 revealed that 8.3% of the U.S. population is diabetic. Diabetes occurs due to insulin secretion abnormality or insulin receptor abnormality, resulting in the inability of regulating the blood glucose levels at a normal level, and complications such as vascular disease and visual loss are present. Representative diabetes therapies include an insulin-dependent therapy and a therapy through lowering blood glucose levels. As conventional materials for treatment through lowering blood glucose levels, biguanide compounds, thiazolidinedione compounds, resveratrol, Coptis chinensis extract, etc., are known. According to a report by the Korean Ministry of Food and Drug Safety, about 470 kinds of domestic diabetes drugs are being distributed after their approval.

Recently, side-effects such as cardiovascular disease, lactic acidosis, etc., have been found in some diabetes drugs including rosiglitazone (U.S. Pat. No. 5,002,953), butformin, metformin, etc., and thus patients who take these drugs must rely on other drugs. Additionally, metformin, the most commonly used diabetes drug, has a high relative dose and thus increases the per capita cost of diabetes treatment. Additionally, examples of diabetes drugs for oral administration include sulfonylureas, etc., but their mechanism with regard to diabetes treatment is to promote insulin secretion, which is different from the method of promoting glucose uptake.

Meanwhile, when there is tissue damage caused by physical injuries or degenerative diseases, various methods such as insertion of various kinds of implants (e.g., artificial joints), administration of cells derived from the corresponding tissue (chondrocytes, muscle cells, etc.) or stem cells, administration of various growth factors capable of promoting cell growth of various kinds of tissues (TGF-β, BMP-2, BMP-4, EGFP, FGF, VEGF, etc.), etc., are used for recovering or restoring the damaged tissue. However, insertion surgery of implants such as artificial joints has problems in that it is very invasive and requires a long recovery time, and in the case where cells derived from the corresponding tissue or stem cells are administered it results in a high cost, and in the case of stem cell administration, side-effects such as cancer development may occur, and in the case of growth factors, it also results in a high cost and has a limited therapeutic effect.

Furthermore, muscular dystrophy, Duchenn muscular dystrophy, Charcot Marie Tooth disease (CMT), Pompe disease, Farbry's disease, spinal muscular atrophy (SMA), amyotrophic lateral sclerosis (ALS), inflammatory myopathy, myasthenia gravis, etc., are major rare intractable muscle disorders that occur in the peripheral nerves and muscles.

Such muscle disorders are caused by various causes such as gene abnormality, muscle damage, muscle atrophy caused by neurodegeneration, complications of metabolic diseases such as diabetes, uremia, etc., and some surgical treatments are possible, but most of them are intractable.

These muscular disorders initially cause sensory dullness or pains, gradually weaken the muscles of the arms and legs, making movement difficult, and severe difficulties in breathing and inability to move may also occur. In the case of muscle disease, cure is almost impossible at present, but when it is diagnosed early, it is aiming to minimize the disorder by delaying the progress of the disease. Clinical trials are currently underway for eteplirsen from Sarepta and drisapersen from GSK, etc., which are test drugs for indications of Duchenn muscular dystrophy. However, in the case of GSK's drisapersen, its approval has been rejected because of toxicity, and no drug has yet been approved for sale.

### SUMMARY

The present disclosure has been made to solve the above-mentioned issues and the present disclosure aims to provide a recombinant exosome which can be used for more efficient regulation of blood glucose levels and regeneration of various tissues including muscles by promoting glucose uptake, and use thereof. However, these are provided for illustrative purposes and the scope of the present disclosure should not be limited thereto.

In this regard, the present disclosure provides a recombinant exosome in which a glucose transporter (hereinafter, GLUT) protein and a membrane fusogenic protein are contained in a membrane thereof.

The present disclosure also provides a composition for treating diabetes containing the recombinant exosome as an active ingredient.

The present disclosure also provides a pharmaceutical composition for lowering blood glucose levels containing the recombinant exosome as an active ingredient.

The present disclosure also provides a pharmaceutical composition for treating muscular disease, which contains the recombinant exosome or a recombinant exosome containing a membrane fusogenic protein in a membrane thereof as an active ingredient.

The present disclosure also provides a composition for promoting tissue regeneration containing the recombinant exosome or a recombinant exosome containing a membrane fusogenic protein in a membrane thereof.

The present disclosure also provides a method for regulating blood glucose levels of a subject including administering the recombinant exosome to the subject in need of the regulation of blood glucose levels.

### BRIEF DESCRIPTION OF THE DRAWINGS

Exemplary embodiments can be understood in more detail from the following description taken in conjunction with the accompanying drawings, in which:

FIG. 1 shows a schematic diagram illustrating a recombinant exosome obtained from cells transfected to express GLUT and vesicular stomatitis virus glycoprotein (hereinafter, VSV-G) of the present disclosure (left); and a process of transferring GLUT to the cell membrane of the target cell by fusing the recombinant exosome with the target cell (right);

FIG. 2A shows a flowchart illustrating the process of isolating a recombinant exosome from the HEK293T cells, which was co-transfected with the VSV-G expression construct and the CD63-GFP fusion protein expression construct, which were constructed to confirm whether a membrane protein was well transferred to the cell membrane of the target cell according to an exemplary embodiment of the present disclosure; FIG. 2B is an image illustrating the results of western blot analysis results confirming the presence of the expression of VSV-G and CD63-GFP with regard to the isolated recombinant exosome; and FIG. 2C is a histogram illustrating the particle size distribution of the isolated recombinant exosome; and FIG. 2D is a transmission electron microscope image of the recombinant exosome;

FIG. 3 shows images with regard to the distribution pattern of the recombinant exosome isolated from the HEK293T cells co-expressing VSV-G protein and CD63-GFP according to an exemplary embodiment of the present disclosure (top) and a recombinant exosome isolated from the HEK293T cells expressing only CD63-GFP as the control group (bottom), photographed after their respective fusion with the HEK293T cells, which are the target cells;

FIG. 4A is a flowchart illustrating the process of isolating a recombinant exosome from the HEK293T cells co-expressing VSV-G protein and GLUT4-GFP according to an exemplary embodiment of the present disclosure; FIG. 4B is an image illustrating the results of western blot analysis results confirming the expression of VSV-G and GLUT4-GFP with regard to the isolated recombinant exosome; FIG. 4C is a histogram illustrating the particle size distribution of the isolated recombinant exosome and a transmission electron microscope image of the exosome; and FIG. 4D is a fluorescence microscope image illustrating the distribution pattern of fluorescence after the fusion with target cells;

FIG. 5A shows a schematic diagram illustrating the process of a single-vesicle imaging assay used to confirm the docking and fusion of a fusogenic exosome with a liposome according to an exemplary embodiment of the present disclosure; FIG. 5B shows the results of flow cytometric analysis illustrating the FRET efficiency according to pH conditions of the fusogenic liposome confirmed by the single-vesicle imaging assay; FIG. 5C shows a graph illustrating the fractions between the low-FRET group, the middle-FRET group, and the high-FRET group as a result of flow cytometry in FIG. 5B; and FIG. 5D is a graph comparing the docking efficiencies according to lipid-mixing with liposomes according to changes in the pH between exosomes having no LDLR and exosomes having LDLR, as receptors of VSV-G;

FIG. 6A is a graph representing the results of glucose uptake levels in a muscle cell line (C2C12) after treating insulin, the fusogenic exosome according to the present disclosure and the control exosome, respectively; FIG. 6B is a histogram illustrating the results of flow cytometry analysis with regard to glucose uptake levels in a muscle cell line (C2C12) after insulin treatment; and FIG. 6C is a histogram illustrating the results of flow cytometry analysis with regard to glucose uptake levels when the muscle cell line (C2C12) was treated with the recombinant exosome obtained from the HEK293T cells co-expressing VSV-G protein and GLUT4-GFP according to an exemplary embodiment of the present disclosure and the control exosome obtained from the HEK293T cells expressing GLUT4-GFP alone;

FIG. 7 shows a graph illustrating the results with regard to the changes in the glucose uptake levels according to 2-NBDG treatment after treating the HEK293T cells pretreated or untreated with apigenin, a GLUT1 expression inhibitor, with the recombinant exosome obtained from the HEK293T cells co-expressing VSV-G protein and GLUT4-GFP according to an exemplary embodiment of the present disclosure and the control exosome obtained from the HEK293T cells expressing GLUT4-GFP alone (*: P<0.05, **: P<0.01, and ***: P<0.001);

FIG. 8A shows a graph illustrating the observation results with regard to the glucose uptake levels by [¹⁸F]-FDG PET radiography after injecting the recombinant exosome of the present disclosure into the leg muscle of an actual animal experiment; FIG. 8B shows images of the PET radiography (*: P<0.05 and **: P<0.01); and FIG. 8C shows images of a confocal immunofluorescence microscope for confirming whether GLUT4 protein is present as a muscle cell membrane according to the presence or absence of VSV-G protein after intramuscular injection of the recombinant exosome of the present disclosure. In FIG. 8A, the vertical axis value of the graph represents SUV, which is the ratio of the radiation dose in the tissue, and is calculated as the radiation dose to be administered (MBq/g) per the radiation dose (MBq/cc)/body weight in the tissue;

FIG. 9A shows a schematic diagram illustrating the administration schedule of the recombinant exosome according to an exemplary embodiment of the present disclosure into the inside of the muscle where damage was induced by CTX; FIG. 9B shows microscope images of the muscle tissue illustrating the effect of the administration of the recombinant exosome on muscle regeneration observed therein; and FIG. 9C shows fluorescence microscope images illustrating the observation results of muscle cell fusion when muscle cells were treated with a fusogenic exosome containing a recombinant exosome and VSV-G according to an exemplary embodiment of the present disclosure.

### DETAILED DESCRIPTION OF EMBODIMENTS

Hereinafter, specific embodiments will be described in detail with reference to the accompanying drawings.

### Definition of Terms

As used herein, the term “glucose transporter” refers to a protein that facilitates the transport of glucose into the cell membrane and into the cell, and the glucose receptor is a membrane protein containing 12 transmembrane helices and both the amino and carboxy terminals are exposed to the cytoplasmic side thereof. So far, 14 kinds of glucose receptors are reported in humans, and according to the amino acid homology, class I (to which GLUT1 to GLUT4, and GLUT14 belong) and class II (to which GLUT5, GLUT7, GLUT9, and GLUT11 belong), and class III (to which GLUT6, GLUT8, GLUT10, GLUT12, and HMIT (H⁺/myoinositol transporter) belong) are included therein.

As used herein, the term “membrane fusogenic protein” refers to a protein that induces a homologous or target fusion between cells or membrane vesicles surrounded by a plasma membrane. The representative membrane fusogenic protein as such may include vesicular stomatitis virus glycoprotein (VSV-G), and may additionally include tat protein of HIV, herpesvirus glycoprotein B (gB) such as HSV-1 gB, EBV gB, thogoto virus G protein, baculovirus gp64 such as AcMNPV gp64, Borna disease virus glycoprotein (BDV G), etc.

As used herein, the term “vesicular stomatitis virus G envelope glycoprotein (VSV-G protein)” refers to a virus glycoprotein that is uniquely present in the virion membrane of vesicular stomatitis virus and it acts as a protein for the attachment of virus to its target cell and fusion thereof. The VSV-G protein is a transmembrane protein containing two N-linked glycans and can initiate a membrane fusion in a low-pH-dependent manner when no other viral proteins are present. Since VSV-G protein forms a complex with nucleic acid molecules such as DNA, etc., it can be used as a carrier for direct gene transfer or for the production of more stable and high-titer pseudotyped murine leukemia virus (MLV)-based retrovirus and lentivirus-based vectors and thus have been used effectively in gene therapy. However, it has recently been proposed that the protein can be used as a target cell for transferring various proteins, in addition to genes, to the target cell (Mangeot et al., Mol. Ther. 19(9): 1656-1666, 2011).

As used herein, the term “exosome” refers to a cell-derived vesicle present in all biological fluids including blood, urine, and cell culture media, and is also called extracellular vesicle or microvesicle. The exosome is known to have a size between 30 nm and 100 nm and when the multivesicular body fuses with a cell membrane, exosome is secreted from the cell or secreted directly through the cell membrane. Exosome is known to play an important role in various processes such as coagulation, intercellular signaling, and metabolic waste management.

As used herein, the term “recombinant exosome” refers to an artificially-produced exosome, and the recombinant exosome is an exosome obtained from the culture broth after culturing a transfected host cell, which was produced by transfecting a host cell capable of producing an exosome with a gene encoding a heterologous protein by genetic engineering. In the recombinant exosome, the transfected foreign protein is contained in the internal or exosome membrane.

In accordance with an aspect of the present disclosure, there is provided a recombinant exosome, in which a glucose transporter (GLUT) protein and a membrane fusogenic protein are contained in a membrane thereof.

The recombinant exosome may be those which are transfected into a first gene construct containing a polynucleotide encoding the GLUT protein and a second gene construct containing a polynucleotide encoding the membrane fusogenic protein and then isolated/purified from mammalian cells, and preferably from human cells, that can overexpress the GLUT protein and the membrane fusogenic protein. Optionally, the recombinant exosome may be those which are transfected into a single gene construct containing both containing the polynucleotide encoding the GLUT protein and the polynucleotide encoding the membrane fusogenic protein and then isolated/purified from mammalian cells, and preferably human cells, that can overexpress the membrane fusogenic protein and the membrane fusogenic protein. The two polynucleotides contained in the single gene construct may be expressed by being operably linked to the same or different promoter or it is possible that they are operably linked to a single promoter and expressed as a single transcript and then separated by internal ribosomal entry sites (IRESs) inserted between the two polynucleotides and translated into their respective proteins.

In the recombinant exosome, the GLUT protein may be GLUT1, GLUT2, GLUT3, GLUT4, GLUT5, GLUT6, GLUT7, GLUT5, GLUT9, GLUT10, GLUT11, GLUT12, H⁺/myoinositol transporter (HMIT), or GLUT14.

In the recombinant exosome, the membrane fusogenic protein membrane fusogenic protein may be vesicular stomatitis virus glycoprotein (VSV-G), tat protein of HIV, HSV-1 gB, EBV gB, thogoto virus G protein, or AcMNPV gp64.

The recombinant exosome may be obtained from a transfected cell for the expression of the GLUT protein and the membrane fusogenic protein so that the GLUT protein and the membrane fusogenic protein can be expressed therein.

The recombinant exosome may further contain at least one therapeutic agent for diabetes. The therapeutic agent for diabetes may be metformin, buformin, phenformin, rosiglitazone, pioglitazone, troglitazone, tolbutamide, acetohexamide, tolazamide, chlorpropamide, glibenclamide, mitiglinide, glipizide, glyburide, glimepiride, gliclazide, glycopiramide, gliquidone, repaglinide, nateglinide, miglitol, acarbose, voglibose, glucagon-like peptide-1 (GLP-1) or a derivative thereof, vildagliptin, stagliptin, saxagliptin, linagliptin, alogliptin, septagliptin, or tenegliptin.

The incorporation of the drug into the recombinant exosome may be achieved by culturing genetically-engineered cells to produce a recombinant exosome in a cell culture medium where the drug is dissolved, and the recombinant exosome may be produced by re-preparing the exosome by adding the isolated recombinant exosome into a solvent where the therapeutic agent for diabetes is dissolved followed by ultrasonic treatment (Kim et al., Nanomedicine, 12(3): 655-664, 2016). Optionally, when the drug is dipped into the exosome, the isolated exosome may be simply mixed with the drug by stirring in an appropriate solvent or medium for a suitable period of time (Sun et al., Mol. Ther. 18(9): 1606-1614, 2010), or in the case of a hydrophilic drug such as nucleic acid, the drug may be incorporated into the exosome by electroporation.

In accordance with an aspect of the present disclosure, there is provided a pharmaceutical composition for treating muscular disease containing the recombinant exosome as an active ingredient.

In accordance with another aspect of the present disclosure, there is provided a pharmaceutical composition for lowering blood glucose levels containing the recombinant exosome as an active ingredient.

In accordance with still another aspect of the present disclosure, there is provided a pharmaceutical composition for treating muscular disease containing the recombinant exosome obtained from a eukaryotic cell transfected for the expression of the recombinant exosome and membrane fusogenic protein as an active ingredient.

In the pharmaceutical composition for treating muscular disease, the muscular disease may be fibromyalgia, inflammatory myopathy, muscular dystrophy, Duchenn muscular dystrophy, Huntington's disease, Parkinson's disease, myalgia, soft tissue sarcoma, polymyalgia rheumatic, muscle cramps, Charcot Marie Tooth disease (CMT), pompe disease, Farbry's disease, Cori's or Forbe's disease, Tarui's disease, McArdie's disease, myositis, inclusion body myositis, Sharp's syndrome, multiple myositis, carpal tunnel syndrome, multiple peripheral neuropathy, spinal muscular atrophy (SMA), amyotrophic lateral sclerosis (ALS), inflammatory myopathy, myasthenia gravis, or muscle tear.

In accordance with still another aspect of the present disclosure, there is provided a composition for promoting tissue regeneration which contains GLUT4 and a membrane fusogenic protein in a membrane thereof.

The composition for promoting tissue regeneration may be used for the regeneration of tissue selected from the group consisting of bone, cartilage, muscle, liver, tendon, ligament, periodont, nerve, lymph, cornea, lung, kidney, large intestine, stomach, small intestine, pancreas, thyroid, and prostate.

The pharmaceutical composition of the present disclosure may contain a pharmaceutically acceptable carrier. The composition containing a pharmaceutically acceptable carrier may be various oral or parenteral formulations, but is preferably a formulation for parenteral administration. For the preparation of formulations, a diluent or excipient such as a filler, an extender, a binder, a humectant, a disintegrant, a surfactant, etc., may be used. Solid formulations for oral administration may include tablets, pills, powders, granules, capsules, etc., and these solid formulations may be prepared by adding at least one excipient, e.g., starch, calcium carbonate, sucrose or lactose, gelatin, etc. Additionally, lubricants, such as magnesium stearate, talc, etc., may be used, in addition to the simple excipient. Liquid formulations for oral administration may include suspensions, liquid medicines for internal use, emulsions, syrups, etc., and various excipients such as humectants, sweeteners, fragrances, and preservatives, may be used, in addition to the frequently-used simple diluents such as water and liquid paraffin. Formulations for parenteral administration may include sterile aqueous solutions, non-aqueous solvents, suspensions, emulsions, lyophilized formulations, suppositories, etc. Examples of the non-aqueous solvents and suspensions may include vegetable oils such as propylene glycol, polyethylene glycol, and olive oil; an injectable ester such as ethyl oleate; etc. Examples of the bases for suppositories may include Witepsol, macrogol, Tween 61, cacao butter, laurinum, glycerogelatin, etc.

The pharmaceutical composition may have one formulation selected from the group consisting of tablets, pills, powders, granules, capsules, suspensions, solutions, emulsions, syrups, sterilized aqueous solutions, non-aqueous solutions, lyophilized formulations, and suppositories.

The pharmaceutical composition of the present disclosure may be administered orally or parenterally. When administered parenterally, the pharmaceutical composition may be administered via various routes, including intravenous injection, intranasal inhalation, intramuscular administration, intraperitoneal administration, transdermal absorption, etc.

The composition of the present disclosure may be administered in a pharmaceutically effective amount.

As used herein, the term “pharmaceutically effective amount” refers to an amount sufficient for the treatment of diseases at a reasonable benefit/risk ratio applicable to a medical treatment, and the level of the effective dose may be determined based on the factors including the kind of a subject, severity of illness, age, sex, drug activity, drug sensitivity, administration time, administration route and dissolution rate, length of treatment, factors including drug(s) to be used simultaneously in combination, and other factors well-known in the medical field. The pharmaceutical composition of the present disclosure may be administered in an amount of 0.1 mg/kg to 1 g/kg, and more preferably, 1 mg/kg to 500 mg/kg. Meanwhile, the administration dose may be appropriately adjusted according to the age, sex, and health conditions of a patient.

The composition of the present disclosure may be administered as an individual therapeutic agent, in combination with other therapeutic agents for diabetes or muscular disease, or sequentially or simultaneously with a conventional therapeutic agent(s), and may be administered once or multiple times. It is important to administer an amount to obtain the maximum effect with a minimum amount without adverse effects considering all of the factors described above, and these factors can easily be determined by one of ordinary skill in the art.

In accordance with still another aspect of the present disclosure, there is provided a method for regulating blood glucose levels of a subject including administering the recombinant exosome to the subject in need of the regulation of blood glucose levels.

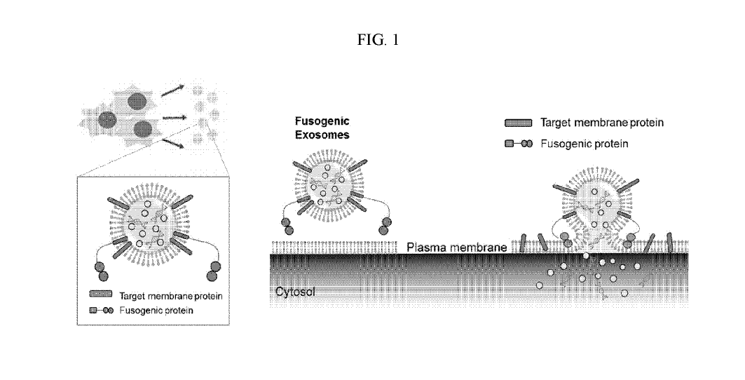

The present inventors have studied on a new biocompatible nano platform using a recombinant exosome to transfer functional membrane proteins to a cell membrane. The present inventors have named this modification of cell membrane as “membrane-editing” (FIG. 1). Exosome is a small membrane vesicle, generally having a diameter of 30 nm to 150 nm and is known to be derived from the multivesicular bodies of parental cells. Due to the possibility of transfer of functional biomolecules (e.g., siRNA, miRNA, mRNA and protein, etc.) between cells and the high ease of manipulation, the therapeutic use of natural or surface-engineered exosomes have recently received attention (Andaloussi et al., Nat. Rev. Drug Discovery 12: 347-357, 2013; Ferguson et al., J. Control. Release 228: 179-190, 2016; Vader et al., Adv. Drug Delivery Rev. 106: 148-156, 2016).

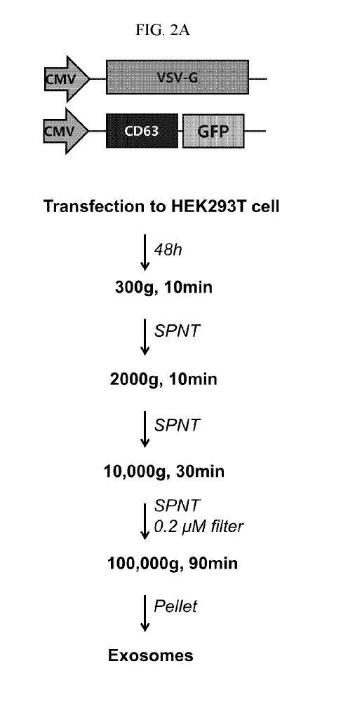

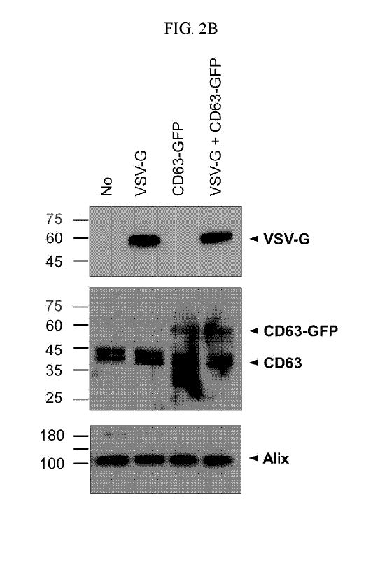

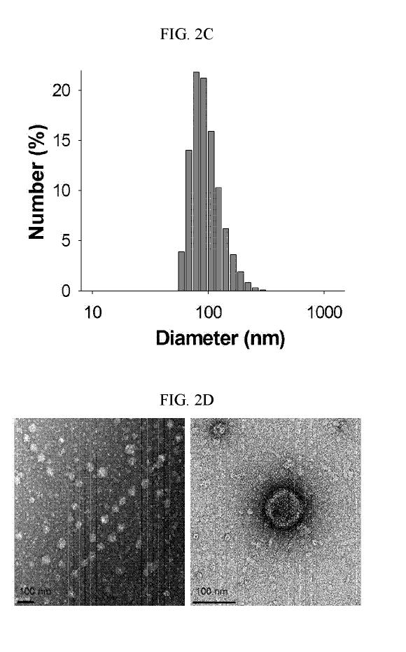

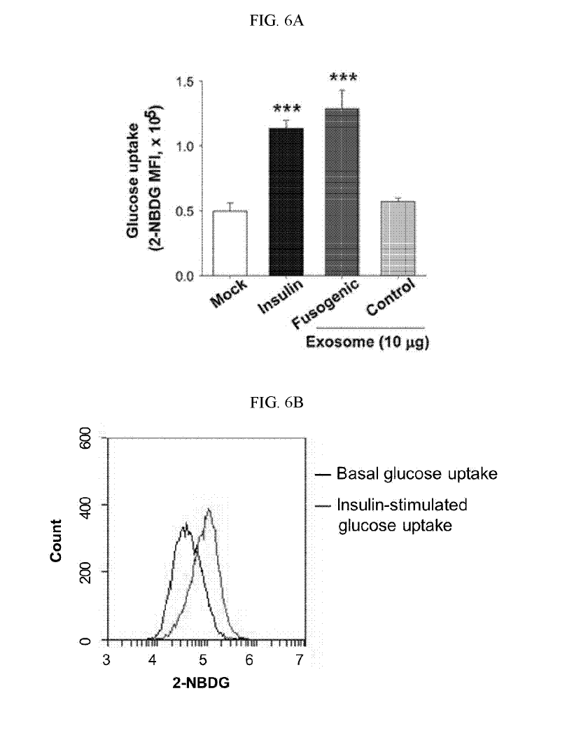

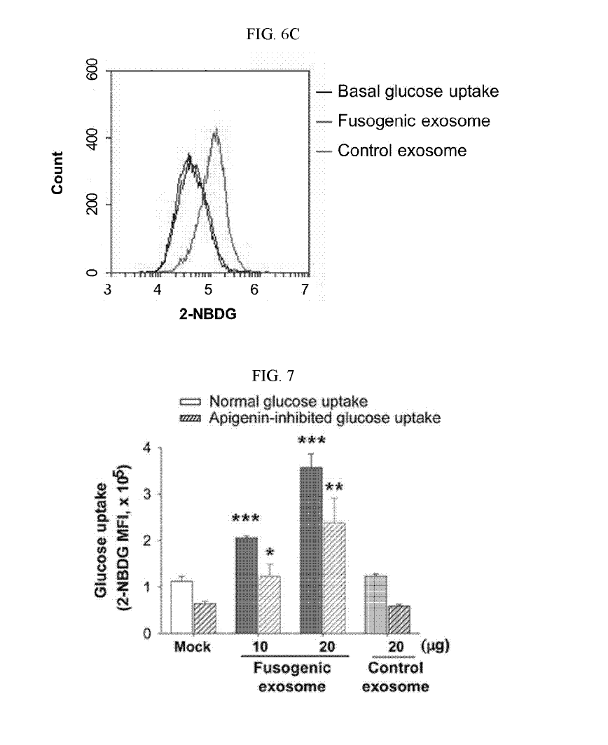

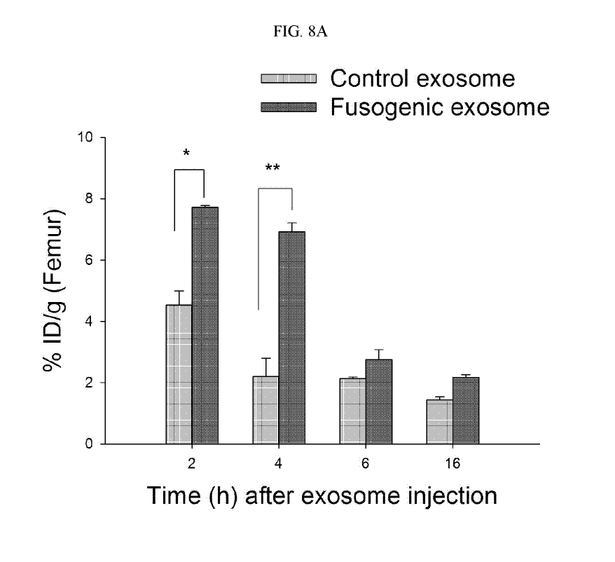

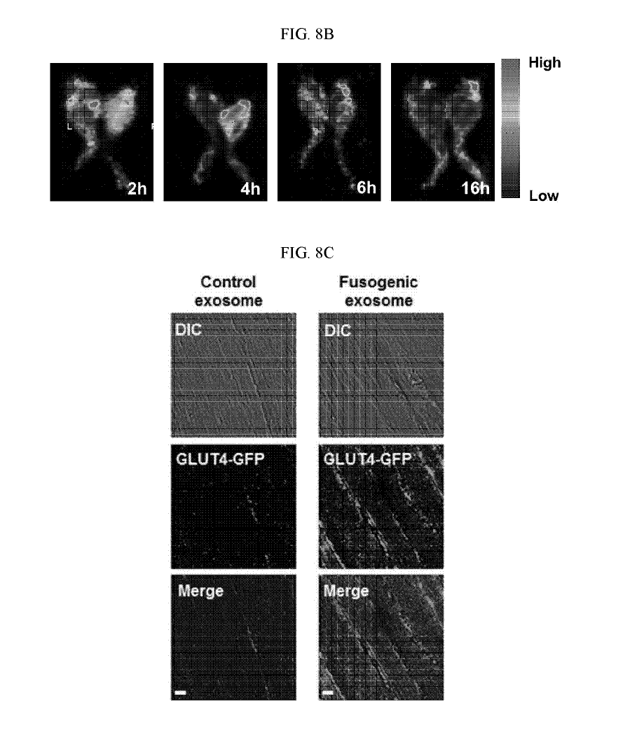

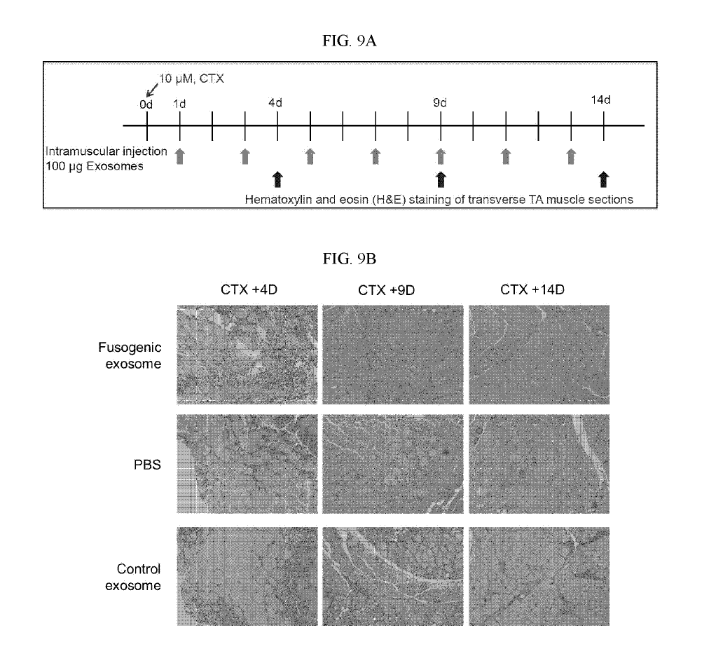

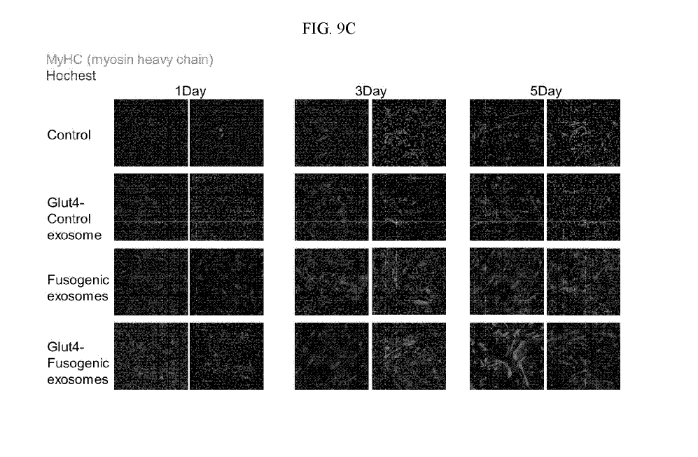

The present inventors have made intensive efforts to develop a technique for efficiently transferring a membrane protein to the cell membrane of cells with a defective membrane using the recombinant exosome. As a result, they have established a hypothesis that when a cell or subject is treated with a recombinant exosome, which was constructed to express the VSV-G (i.e., a viral fusogen) on the surface of a cell or subject by loading a corresponding membrane protein to be transferred thereon, the corresponding membrane protein to be transferred can be efficiently transferred to the cell or subject (FIG. 1). For confirming the hypothesis, the HEK293T cells, which were transfected for the expression of the VSV-G protein (i.e., a membrane fusogenic protein) and CD63 or GLUT-4 protein (i.e., a target membrane protein), were cultured and the exosomes secreted therefrom were isolated and purified and thereby it was confirmed that the target membrane protein was normally located on the surface of the exosomes (FIGS. 2A to 4D). Additionally, it was confirmed that when the HEK293T cells were treated with the recombinant exosome, in which VSV-G protein and GLUT-4 were contained therein, the glucose uptake rate was significantly increased compared to that of the control group (FIGS. 6A to 7). Accordingly, it was confirmed that the recombinant exosome according to an exemplary embodiment of the present disclosure was able to fuse with the membrane of the target cell so that that it could effectively transfer the target membrane protein to the cell membrane of the target cell. Furthermore, it was confirmed that when the VSV-G protein or the recombinant exosome, in which VSV-G protein and GLUT-4 were contained therein, according to an exemplary embodiment of the present disclosure was directly administered to the damaged area of the animal model, in which muscle damage was induced, the muscle damage was more rapidly recovered compared to that of the control group, and such ability of tissue regeneration was due to not only the promotion of glucose uptake through GLUT-4 transferred to the cell membrane of the damaged tissue by the recombinant exosome according to an exemplary embodiment of the present disclosure, but also the promotion of the fusion of muscle cells according to the activity of membrane fusion by VSV-G protein (i.e., a fusotropic effect) (FIGS. 8A to 9C). These results suggest that the recombinant exosome according to an exemplary embodiment of the present disclosure may be utilized for the regeneration of various tissues such as bones, ligaments, cartilages, muscles, etc., as well as regulation of blood glucose levels.

Furthermore, the recombinant exosome of the present disclosure may be utilized as a very useful platform technology for an efficient transfer of other therapeutic membrane proteins, in addition to the GLUT-4, to a target cell and organ.

### BEST MODE FOR CARRYING OUT THE INVENTION

Hereinafter, preferred embodiments of the present disclosure will be described in detail. However, the present disclosure is not limited to embodiments explained herein but may be specified in various aspects. Rather, the embodiments are provided to sufficiently transfer the concept of the present disclosure to a person skilled in the art to thorough and complete contents introduced herein.

### Example 1: Preparation of Recombinant Exosome Containing VSV-G and CD63 and Introduction into Target Cell

The present inventors have proposed a hypothesis that when a membrane protein is incorporated into an exosome containing a VSV-G protein and put into contact with a target cell, the membrane protein can be transferred into the cell membrane of the target cell (see FIG. 1). To investigate the actual feasibility of the hypothesis, they introduced a fusion protein where a GFP (a fluorescent protein) was fused with CD63 (when a membrane protein when a membrane protein), into an exosome and the distribution pattern of fluorescence after the contact with the target cell was observed.

Specifically, the present inventors purchased the pCMV-VSV-G Envelope Vector (RV-110, Cell Biolabs, hereinafter, “VSV-G construct”), where the gene encoding the VSV-G protein (SEQ NO ID: 1) is inserted, and purchased the pCT-CD63-GFP vector (hereinafter, “CD63-GFP construct”), which localizes the CD63-GFP fusion protein into an exosome, from the System Bioscience (San Francisco, USA). The two gene constructs were co-transfected into HEK293T cells and culture for 48 hours. Then, the cell culture was collected and sequentially centrifuged at 300×g for 10 minutes, at 2,000×g for 10 minutes, and 10,000×g for 30 minutes, filtered using a 0.2 μm filter, and then subjected to ultrafiltration at 100,000×g for 90 minutes to recover pellets (FIG. 2A).

To confirm whether the VSV-G protein and CD63-GFP were included in the exosome, part of the recovered exosome was disrupted and western blot analysis was performed using anti-VSV-G antibody (Abcam, ab50549), anti-CD63 antibody (Abcam, ab8219), anti-Alix antibody (exosome markers, Santacruz, sc99010) (FIG. 2B). As a result, as shown in FIG. 2B, only CD63 was detected in the exosome derived from non-transfected HEK293T cells (control group), whereas only VSV-G protein and CD63 were detected in the exosome derived from the HEK293T cells transfected with only the VSV-G construct, only CD63 and CD63-GFP were detected in the exosome derived from the HEK293T cells transfected with only the CD63-GFP construct, and all of VSV-G, CD63, and CD63-GFP were detected in the exosome derived from the HEK293T cells co-transfected with both the VSV-G construct and the CD63-GFP construct. These results suggest that the VSV-G and the membrane protein are normally included in the exosomes derived from the cells transfected for the expression of the VSV-G and the membrane protein according to an exemplary embodiment of the present disclosure.

Then, the present inventors analyzed the size distribution of the recovered exosomes by dynamic light scattering (DLS) with a Zetasizer nano ZS (UK) (see FIG. 2C) and the exosomes produced were photographed under a transmission electron microscope (see FIG. 2D). As a result, as shown in FIGS. 2C AND 2D, the exosomes prepared according to an exemplary embodiment of the present disclosure were shown to have a very narrow spectrum with a size of around 100 nm.

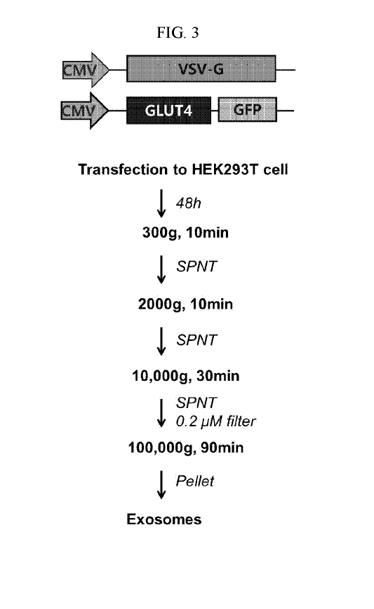

Then, the present inventors treated the exosomes obtained above in the HEK293T cells and examined to locate the distribution of the membrane protein (CD63-GFP) present in the exosomal membrane by a fluorescence microscope (see FIG. 3). Meanwhile, to confirm whether the membrane protein is located on the cell membrane, the cell membrane of the HEK293T cells was pre-stained with RFP by transfecting the cells using the CellLight™ Plasma Membrane-RFP (BacMam 2.0, Thermo Fisher Scientific).

As a result, as shown in FIG. 3, in the case of the cells treated with fusogenc exosomes (exosomes obtained from the cells transfected for the expression of both VSV-G protein and CD63-GFP), the distribution by RFP with pre-stained cell membrane agreed with the distribution of green fluorescence by GFP thus confirming that CD63-GFP was properly transferred into the cell membrane of the target cells. Meanwhile, in the case of the cells treated with control exosomes obtained from the cells expressing only CD63-GFP without VSV-G, it was confirmed that CD63-GFP was distributed in the early endosomes within the cells thus confirming that it was captured into the cells by endocytosis.

### Example 2: Preparation of Recombinant Exosome Containing VSV-G and GLUT4 and Introduction into Target Cell

The present inventors, from the result of Example 1, have attempted to confirm whether the specific transfer of the target cells of GLUT4 (i.e., a glucose transporter), to a cell membrane and the subsequent glucose uptake in the target cells and target tissues by the transfer are possible.

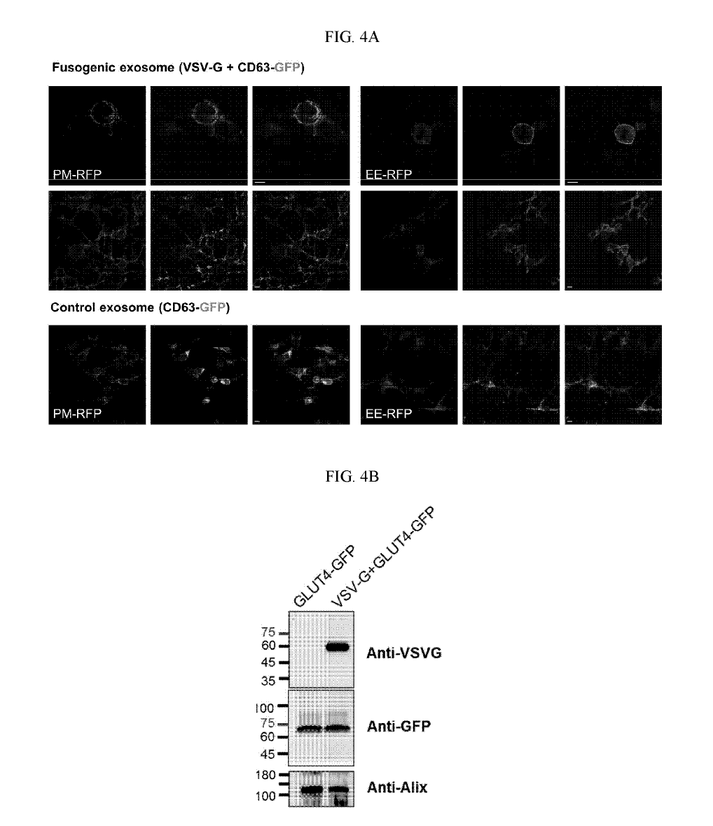

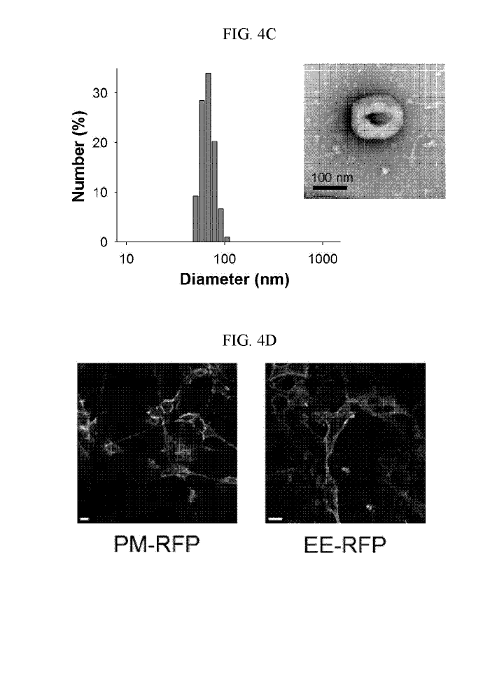

Specifically, the present inventors purchased HG13123-ACG (Sino Biological), which is a GLUT4-GFP fusion construct where a polynucleotide encoding GFP was linked to 3′-end of the polynucleotide encoding the human GLUT4. After co-transfecting the GLUT4-GFP construct and the VSV-G construct prepared in Example 1 into HEK293T cells and culture for 48 hours. Then, the cell culture was collected and sequentially centrifuged at 300×g for 10 minutes, at 2,000×g for 10 minutes, and 10,000×g for 30 minutes, filtered using a 0.2 μm filter, and then subjected to ultrafiltration at 100,000×g for 90 minutes to recover pellets (FIG. 4A).

Then, to confirm whether the VSV-G protein and GLUT4-GFP were included in the exosome, part of the recovered exosome was disrupted and western blot analysis was performed using anti-VSV-G antibody (Abeam, ab50549) and anti GFP antibody (Abcam, ab290) (FIG. 4B). As a result, as shown in FIG. 4B, only GLUT4-GFP was detected in the exosome derived from the HEK293T cells transfected with GLUT4-GFP construct, whereas both VSV-G and GLUT4-GFP were detected in the exosome derived from the HEK293T cells co-transfected with both the VSV-G construct and the GLUT4-GFP construct.

Then, the present inventors analyzed the size distribution of the recovered exosomes and the exosomes produced were photographed under a transmission electron microscope (see FIG. 4C). As a result, as shown in FIG. 4C, the exosomes prepared according to an exemplary embodiment of the present disclosure were shown to have a no distinct difference in size compared to the result of Example 1.

From the above results, the present inventors treated the exosomes obtained above in the C2C12 cells (target cells) to examine whether GLUT4 protein is well transferred to the cell membranes of the target cells when the exosomes were treated un the target cells, and the intracellular distribution pattern of GLUT4 was confirmed under a fluorescence microscope (see FIG. 4D). As a result, as shown in FIG. 4D, it was confirmed that when the exosomes obtained from the cells co-transfected with VSV-G construct and GLU4-GFP construct were treated in the C2C12 cells, the fluorescence distribution by GFP was co-localized with PM-RFP (a cell membrane marker) but did not agree with the fluorescence distribution by EE-RFP (an endosome marker).

### Experimental Example 1: Evaluation of Ability of Exosome Fusion

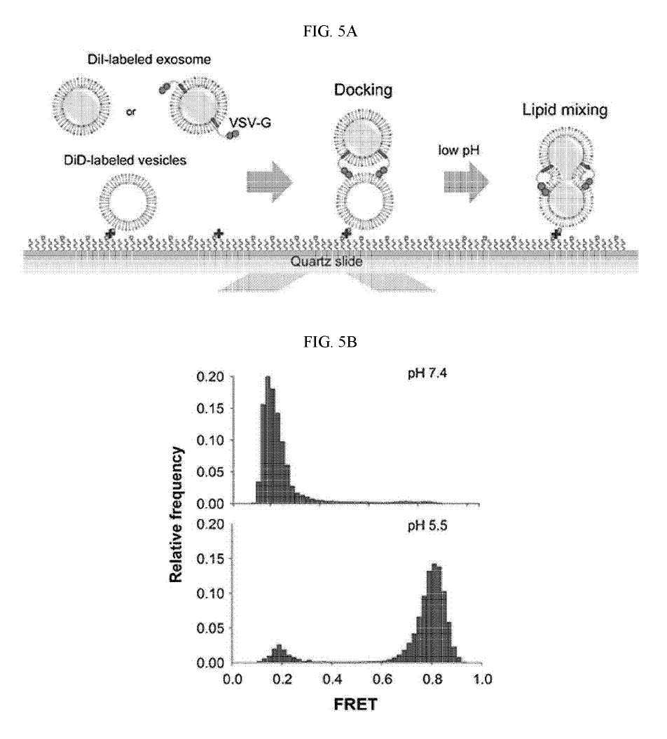

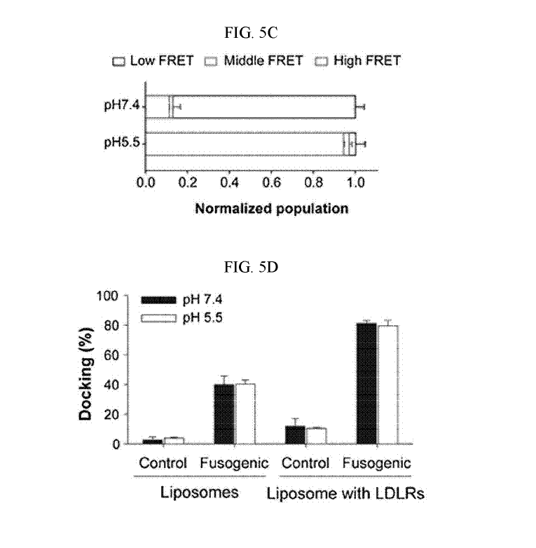

To confirm whether the transfer of membrane proteins are attributed to the fusogenic activity of VSV-G contained in the recombinant exosome prepared in Example 1, the present inventors have developed an in vitro single-vesicle imaging assay to monitor the fusion efficiency of single exosome particles with artificial liposomes that mimic the cellular membrane lipid composition. Specifically, the present inventors have performed a fluorescence resonance energy transfer (FRET) assay that can provide an average of the number of fusion events between exosomes and artificial liposomes using a total internal reflection fluorescence microscope. To this end, liposomes were doped with 1% nitrilotriacetic acid (NTA) lipids to bind His-tagged low-density lipoprotein receptors (LDLRs), which serve as the cellular entry port of VSV-G. Recent studies showed that VSV-G binds universal cell surface LDLRs and other LDL family members to enable binding of VSV and fusion with target cell membranes. In the single-vesicle imaging assay, vesicles formed pairs with any one among different vesicles having donor donor fluorophore (DiI) and acceptor fluorophore (DiD) (FIG. 5A). FIG. 5A shows a schematic diagram illustrating the process of a single-vesicle imaging assay according to an exemplary embodiment of the present disclosure. FRET efficiency was measured after each pair of vesicles has equilibrated and the unpaired vesicles had been removed. The data from the paired vesicles were used to distinguish between docked (low FRET) and fully lipid-mixed populations (high FRET). As shown in FIG. 5A, in the flow cells of flow cytometer, DiD fluorophore-labeled liposomes were immobilized on the PEG-coated surface, donor (DiI-labeled) exosomes were added, and the formation of single exosome-liposome complexes triggered lipid mixings in an acidic condition of pH 5.5, thereby increasing FRET efficiency such as about 86% of docked vesicles were lipid-mixed, etc. (FIG. 5B lower panel and FIG. 5C). In FIG. 5C, low-FRET indicates when the FRET efficiency is below 0.4, middle-FRET indicates when the FRET efficiency is 0.4 or higher and below 0.65, and high-FRET indicates when the FRET efficiency is 0.65 or higher. In contrast, at neutral pH, few exosome-liposome complexes showed lipid mixing after 30 minutes of incubation (FIG. 5B, upper panel). Furthermore, the present inventors have confirmed that LDLR could stimulate the docking between liposomes and exosomes, which is VSV-G-dependent (FIG. 5D and Table 1). FIG. 5D is a graph illustrating the fractions of the docking subpopulations. These results suggest that the components of exosomes are not sufficient to achieve stable exosome binding.

[symbol] Table 1 [symbol] Number of docking to lipid-mixing and total number of liposomes

| | | | Docked | Biotinylated |
|---|---|---|---|---|
| pH | DiI Channel | DiD Channel | exosome^{a} | Liposome^{b} |
| 7.4 | Control exosome | Liposome | 6.9 | 257 |
| | Fusogenic exosome | | 84 | 211 |
| 5.5 | Control exosome | | 8 | 202.7 |
| | Fusogenic exosome | | 96 | 238.4 |
| 7.4 | Control exosome | Liposome with | 33 | 281 |
| | Fusogenic exosome | LDLR | 169 | 208.4 |
| 5.5 | Control exosome | | 28 | 267 |
| | Fusogenic exosome | | 180 | 226.6 |

^{a}DiI-labeled exosome were flowed into flow cells to allow vesicle docking.
^{b}labeled liposomes were immobilized on the PEG-coated surface of the flow cell through the streptavidin-to-biotin lipid conjugation.

### Experimental Example 2: Evaluation of In Vitro Glucose Uptake Ability

From the results of Example 2, the present inventors have investigated whether the glucose uptake ability of target cells can be increased by the treatment with the recombinant exosome according to an exemplary embodiment of the present disclosure.

Specifically, the present inventors have seeded muscle cell line C2C12 cells, which are target cells, to a 24-well plate at a concentration of 10⁵ cells/mL, and the medium was replaced with glucose- and serum-free DMEM medium, and treated with exosomes (20 μg/mL) obtained in Example 2 or insulin (1 μg/mL) for 1 hour. Then, the resultant was treated with 2-(N-(7-nitrobenz-2-oxa-1,3-diazol-4-yl)amino)-2-deoxyglucose (2-NBDG) (150 μg/mL), which is a glucose targeting fluorescent ligand, and the presence of glucose uptake was measured by flow cytometry (excitation light: 488 nm) using a glucose uptake cell-based assay kit (Cayman) (see FIGS. 6A to 6C).

As a result of the above assay, as shown in FIGS. 6A to 6C, most of the C2C12 cells treated with fusogenic exosomes (containing VSV-G protein and GLUT4-GFP) showed a similar level of increase in fluorescence value by treatment with 2-NBDG to that by insulin treatment, whereas the C2C12 cells treated with non-fusogenic exosomes (containing only GLUT4-GFP) showed a fluorescence value by treatment with 2-NBDG equivalent to that of control cells (without exosome treatment). These results suggest that it is difficult to improve glucose uptake ability by exosomes containing GLUT4 alone.

In this regard, in order to investigate whether these results are actually due to the effect by the transfer of GLUT4 into cell membranes, the present inventors analyzed the change in the level of glucose uptake when glucose uptake was suppressed by apigenin, a GLUT1-specific inhibitor.

Specifically, the present inventors seeded the HEK293T cells (i.e., target cells) into a 24-well plate at a concentration of 6×10⁴ cells/mL and then replaced the medium with glucose- and serum-free medium. Then, the HEK293T cells were pretreated with 100 μM of apigenin, an inhibitor of GLUT1 expression, 4 hours before the treatment with exosomes. Subsequently, the HEK293T cells were treated with fusogenic exosomes prepared in Example 2 at concentrations of 10 μg/mL and 20 μg/mL, and with non-fusogenic exosomes as a control group at a concentration of 20 μg/mL, and 30 minutes thereafter treated with 2-NBDG at a concentration of 150 μg/mL, and the presence of glucose uptake was measured by flow cytometry (excitation light: 488 nm) using glucose uptake cell-based assay kit (Cayman Chemical, USA) (see FIGS. 7A and 7B.).

As a result, as shown in FIGS. 7A and 7B, the cells treated with fusogenic exosomes according to an exemplary embodiment of the present disclosure showed an increase in the glucose uptake rate in an exosome concentration-dependent manner, and when the cells were treated with apigenin, the glucose uptake rate was slightly decreased but the group where the cells were treated with 20 μg/mL of apigenin showed a higher glucose uptake rate compared to that of the negative control group, where no treatment was made, and thus it was confirmed that glucose uptake rate was increased despite the suppression of GLUT1 expression and this suggests that GLUT4 was transferred into the cell membranes of target cells by exosomes thereby enabling glucose uptake into the cells.

### Experimental Example 3: Evaluation of In Vivo Glucose Uptake Ability

In this regard, the present inventors performed an animal experiment to confirm whether the administration of the exosomes according to an exemplary embodiment of the present disclosure can actually improve the glucose uptake ability of the corresponding animal.

Specifically, BALB/c-nu mice were injected intramuscularly with 100 μg each of the fusogenic exosomes prepared in Example 2 and the non-fusogenic exosomes on the left and right thighs and PET images were obtained 2-, 4-, 6-, and 16 hours post-injection. 60 minutes prior to PET imaging, the mice were anesthetized with 2% isoflurane and then injected with [¹⁸F]2-fluoro-2-deoxy-D-glucose ([¹⁸F]FDG) at a radiation dose of 7.3-8 MBq via the tail vein, and PET images were obtained using a small animal PET apparatus (nanoScanPET/MRI system 1T, Mediso, Hungary).

As a result, as shown in FIGS. 8A and 8B, it was confirmed that when the fusogenic exosomes according to an exemplary embodiment of the present disclosure was administered, the glucose uptake ability was improved within the muscle, and in particular, it was shown that the glucose uptake efficiency reached the maximum level 4 hours after the administration of exosomes, and a significant difference was maintained until 6 hours after the administration of exosomes.

Additionally, to confirm whether the GLUT4 protein, which was transferred by the fusogenic exosomes according to an exemplary embodiment of the present disclosure, can exhibit correct cell membrane localization in vivo, the present inventors injected the mice intramuscularly with fusogenic exosomes containing the GLUT4-GFP fusion protein prepared in Example 2, and 4 hours thereafter, obtained tibialis anterior (TA) muscle fibers, and performed confocal immunofluorescence microscopy (FIG. 8C). As a result, as shown in FIG. 8C, the muscle fibers treated with control exosomes (exosomes which contain GLUT4-GFP but do not contain VSV-G) exhibited rather weak GFP signals (FIG. 8C, left), whereas the muscle fibers treated with fusogenic exosomes showed strong GFP signals on the surface membranes of TA muscle (FIG. 8C, right). These experimental results confirm that the fusogenic exosomes according to an exemplary embodiment of the present disclosure can not only efficiently transfer GLUT protein to the cell membrane of target cells and target tissues but also improve glucose uptake ability by the same. Accordingly, the composition according to an exemplary embodiment of the present disclosure can be very effectively used for the regulation of blood glucose levels of diabetic patients.

### Experimental Example 4: Evaluation of Effect of Muscle Regeneration

4-1: Effect of Promoting Regeneration of Damaged Muscle Induced by Toxin

The present inventors have confirmed from the results of Example 4 that the recombinant exosome according to an exemplary embodiment of the present disclosure can appropriately transfer GLUT4 to muscle cells in vivo conditions. When muscle damage is induced by the administration of cardiotoxin (CTX) to the tibialis anterior muscle of an experimental animal, the damage can be naturally cured in about 2 to 3 weeks. The present inventors attempted to confirm whether the transfer of GLUT4 into muscle cells by exosomes can further promote muscle regeneration (Moreno H et al., J. Biol. Chem., 2003, 278(42): 40557-40564). Specifically, as reported by Lee et al., 8 to 10 week old C57BL/6 male mice were injected intramuscularly on the tibialis anterior muscle with 50 μL of 10 μM cardiotoxin (CTX), which is a muscle toxin to induce muscle damage (Lee et al., Scientific Reports, 5: 16523, 2015). On the 1st day of the CTX injection, 100 μg each of fusogenic exosomes and non-fusogenic exosomes as control exosome was respectively injected intramuscularly and 100 μg of each of the exosomes was injected intramuscularly 6 additional times at 2 day intervals (FIG. 9A).

On the 4^{th}, 9^{th} and 14^{th} day of the CTX injection, the experimental animals were sacrificed and the tibialis anterior muscles on both sides were obtained, embedded in an OCT compound, sections were prepared to a thickness of 4 μm to 6 μm, and the prepared sections were stained with hematoxylin and eosin, and subjected to histological analysis with a microscope (FIG. 9B). As a result, as shown in FIG. 9B, it was confirmed that the damaged muscle was more rapidly recovered when treated with the fusogenic exosomes of the present disclosure compared to when treated with non-fusogenic exosomes.

4-2: Effect on Fusion of Muscle Cells

Further to the analysis of Experimental Example 4-1, the present inventors have examined the effect of the fusogenic exosomes containing VSV-G according to an exemplary embodiment of the present disclosure on the fusion of muscle cells.

Specifically, muscle cell line C2C12 cells were seeded into a 24-well plate at a concentration of 10⁵ cells/mL and the medium was replaced with glucose- and serum-free DMEM medium, and treated with control exosomes (exosomes which do not contain any of VSV-G and GLUT), non-fusogenic exosomes which contain only GLUT4 but do not contain VSV-G (Glut4-Control exosomes), fusogenic exosomes which contain only VSV-G but do not contain GLUT4, and fusogenic exosomes (Glut4-fusogenic exosomes) which contain both GLUT4 and VSV-G obtained in Example 2, at a concentration of 20 μg/mL, respectively. Then, on the 1^{st}, 3^{rd}, and 5^{th} day, an immunofluorescence analysis was performed using antibodies (DSHB, MF-20) which specifically bind to myosin heavy chain. As a result, as shown in FIG. 9C, no significant difference was observed in the muscle bundle treated with control exosomes and non-fusogenic exosomes containing GLUT4, but those treated with fusogenic exosomes showed an increase in the size of the muscle bundle. These results show that the treatment of the fusogenic exosomes of the present disclosure alone can promote the fusion of muscle cells, and it is thus determined that the VSV-G present on the surface of the fusogenic exosomes can not only induce the fusion of the exosomes into the muscle cells but also induce the fusion between muscle cells. Accordingly, the present inventors have named the fusogenic exosomes of the present disclosure capable of promoting the fusion of muscle cells as fusotrophic exosomes. Furthermore, in the case of fusogenic exosomes containing GLUT4, it was confirmed that the effect of increasing glucose uptake by GLUT4 and the fusotropic effect by VSV-G are added together thereby further promoting the fusion of muscle cells.

These results suggest that the recombinant exosome according to an exemplary embodiment of the present disclosure can be effectively used for the regeneration of damaged muscle, and the recombinant exosome according to an exemplary embodiment of the present disclosure can be effectively used not only for the treatment of various kinds of physical injuries but also for the treatment of intractable muscular disorders such as muscle weakness due to degenerative neurological diseases such as Parkinson's disease or Huntington's disease, and amyotrophic lateral sclerosis.

The present disclosure has been described with reference to embodiments, but it is to be understood that they are provided herein for illustrative purposes and various changes and equivalent embodiments are possible without departing from the scope of the present disclosure by those skilled in the art. Accordingly, the true scope of protection of the present disclosure should be determined by the technical concept of the appended claims.

According to an exemplary embodiment of the present disclosure constituted as described above, the present disclosure can easily regulate the increased blood glucose levels of a subject without resorting to a complex mechanism such as gene transfer. Accordingly, the present disclosure can not only be very effectively used for the regulation of blood glucose levels of patients with type 2 diabetes as well as those with type 1 diabetes, but also can be very effectively used for the regeneration of damaged tissue because the present disclosure can promote regeneration of various kinds of tissues.

1. A recombinant exosome, wherein a glucose transporter (GLUT) protein and a membrane fusogenic protein are comprised in a membrane thereof.

2. The recombinant exosome of claim 1, wherein the GLUT protein is GLUT1, GLUT2, GLUT3, GLUT4, GLUT5, GLUT6, GLUT7, GLUT8, GLUT9, GLUT10, GLUT11, GLUT12 H⁺/myoinositol transporter (HMIT), or GLUT14.

3. The recombinant exosome of claim 1, wherein the membrane fusogenic protein is vesicular stomatitis virus glycoprotein (VSV-G), tat protein of HIV, HSV-1 gB, EBV gB, thogoto virus G protein, or AcMNPV gp64.

4. The recombinant exosome of claim 1, which is obtained from a transfected cell for the expression of the GLUT protein and the membrane fusogenic protein.

5. The recombinant exosome of claim 1, further comprising at least one additional therapeutic agent for diabetes.

6. The recombinant exosome of claim 5, wherein the therapeutic agent for diabetes is metformin, buformin, phenformin, rosiglitazone, pioglitazone, troglitazone, tolbutamide, acetohexamide, tolazamide, chlorpropamide, glibenclamide, mitiglinide, glipizide, glyburide, glimepiride, gliclazide, glycopiramide, gliquidone, repaglinide, nateglinide, miglitol, acarbose, voglibose, glucagon-like peptide-1 (GLP-1) or a derivative thereof, vildagliptin, stagliptin, saxagliptin, linagliptin, alogliptin, septagliptin, or tenegliptin.

7. A pharmaceutical composition for treating diabetes comprising the recombinant exosome according to claim 1 as an active ingredient.

8. A pharmaceutical composition for lowering blood glucose levels comprising the recombinant exosome according to claim 1 as an active ingredient.

9. A pharmaceutical composition for treating muscular disease comprising the recombinant exosome according to claim 1, or a recombinant exosome comprising a membrane fusogenic protein in a membrane thereof as an active ingredient.

10. The pharmaceutical composition according to claim 9, wherein the membrane fusogenic protein is vesicular stomatitis virus glycoprotein (VSV-G), tat protein of HIV, HSV-1 gB, EBV gB, thogoto virus G protein, or AcMNPV gp64.

11. The pharmaceutical composition according to claim 9, wherein the muscular disease is fibromyalgia, inflammatory myopathy, muscular dystrophy, Duchenn muscular dystrophy, Huntington's disease, Parkinson's disease, myalgia, soft tissue sarcoma, polymyalgia rheumatic, muscle cramps, Charcot Marie Tooth disease (CMT), pompe disease, Farbry's disease, Cori's or Forbe's disease, Tarui's disease, McArdie's disease, myositis, inclusion body myositis, Sharp's syndrome, multiple myositis, carpal tunnel syndrome, multiple peripheral neuropathy, spinal muscular atrophy (SMA), amyotrophic lateral sclerosis (ALS), inflammatory myopathy, myasthenia gravis, or muscle tear.

12. A composition for promoting tissue regeneration comprising the recombinant exosome according to claim 1, or a recombinant exosome comprising a membrane fusogenic protein in a membrane thereof.

13. The composition of claim 12, wherein the composition is used for the regeneration of tissue selected from the group consisting of bone, cartilage, muscle, liver, tendon, ligament, periodont, nerve, lymph, cornea, lung, kidney, large intestine, stomach, small intestine, pancreas, thyroid, and prostate.

14. A pharmaceutical composition for treating diabetes comprising the recombinant exosome according to claim 2 as an active ingredient.

15. A pharmaceutical composition for treating diabetes comprising the recombinant exosome according to claim 3 as an active ingredient.

16. A pharmaceutical composition for treating diabetes comprising the recombinant exosome according to claim 4 as an active ingredient.

17. A pharmaceutical composition for treating diabetes comprising the recombinant exosome according to claim 5 as an active ingredient.

18. A pharmaceutical composition for treating diabetes comprising the recombinant exosome according to claim 6 as an active ingredient.

19. A pharmaceutical composition for lowering blood glucose levels comprising the recombinant exosome according to claim 2 as an active ingredient.

20. A pharmaceutical composition for lowering blood glucose levels comprising the recombinant exosome according to claim 3 as an active ingredient.
